(19) United States
(12) Patent Application Publication
(10) Pub. No.: US 2002/0137908 A1
(43) Pub. Date: Sep. 26, 2002

(54) Novel guanosine triphosphate (GTP) binding protein-coupled receptor protein, BG3

(76) Inventors: Takao Nakamura, Ibaraki (JP); Masataka Ohta, Ibaraki (JP)

Correspondence Address: JANIS K. FRASER, PH.D., J.D., Fish & Richardson P.C., 225 Franklin Street, Boston, MA, 02110-2804, US

(21) Appl. No.: 09/963,766

(22) Filed: Sep. 25, 2001

Related U.S. Application Data
(63) Continuation-in-part of application No. PCT/JP00/01826, filed on Mar. 24, 2000.

(30) Foreign Application Priority Data
Mar. 25, 1999 (JP) 11/82641

(51) Int. Cl. C07H 21/02; C07H 21/04

(52) U.S. Cl. 536/23.1

(57) ABSTRACT

The present inventors isolated a cDNA which encodes a novel human G protein-coupled receptor protein, by deducing a unique region highly conserved among known G protein-coupled receptor proteins and screening cDNAs by using the probe corresponding to the region. The G protein-coupled receptor protein of the present invention can be used to screen for ligands and candidate pharmaceutical compounds capable of modulating the signal transduction mediated by the receptor.

This application is a continuation-in-part of PCT/JP00/01826, filed Mar. 24, 2000, and claims priority from Japanese Patent Application No. 11/82641, filed Mar. 24, 1999.

### TECHNICAL FIELD

The present invention relates to a novel G protein-coupled receptor protein that is expressed in different tissues, DNA encoding the protein, methods for producing the protein, and methods of using the protein and DNA.

### BACKGROUND

G protein-coupled receptors (guanosine triphosphate binding protein-coupled receptors) are ubiquitously expressed in mammals, and are thought to play an important biological role.

Generally, G protein-coupled receptor proteins share amino acid sequence similarities to some degree and form several receptor superfamilies. For example, a group of receptor families represented by Class B have peptide ligands and are involved in intracorporeal regulation dependent on various hormones.

α-Latrotoxin receptor is known as one of such G protein-coupled receptor proteins (Valery, G. K. et al., Neuron 18: 925-937 (1997)). α-Latrotoxin is a neurotoxin present in the venome gland of black widow spiders, and it stimulates neurotransmitter release by acting on the presynaptic membrane of the nerve ending. α-Latrotoxin causes depletion of acetylcholine at the neuromuscular junction and facilitates the release of adrenaline from sympathetic nerve terminals, thereby resulting in a number of symptoms including topical pyrexia, pain, swelling of the regional lymph node, abdominal muscle rigidity, emesis, hypertension, tachycardia, excessive perspiration on the face, dysapnea and logopathy. The mortality rate due to this toxin reaches 3 to 12%.

As inferred from the neurotoxic action of a-Latrotoxin, α-Latrotoxin receptors may have peptide ligands and may play a role in secretion of certain neurotransmitters. Furthermore, judging from the suggested involvement of α-Latrotoxin receptors in promoting insulin secretion and such (Jochen, L. et al., EMBO J. 17:648-657 (1998)), α-Latrotoxin receptors are not only involved in neurotransmitter secretion, but are also thought to be receptor proteins involved in secretion of hormones that are widely related to intracorporeal regulation.

One of the pathways that regulates biological functions by means of hormones, neurotransmitters and G protein-coupled receptors is the hypothalamo-hypophysial system. In this system, hypothalamic hormones regulate the secretion of pituitary hormones from the pituitary gland, and the pituitary hormones released in the blood mediate the functional regulation of target cells and organs. For instance, functional regulations that are essential for living organisms, such as maintenance of homeostasis and regulation of the development and growth of the reproductive system and individuals, are conducted by way of this pathway.

Representative hypothalamic hormones are TRH, CRF, GRF, somatostatin, and such, and representative pituitary hormones are TSH, ACTH, FSH, LH, prolactin, growth hormone, oxytocin, vasopressin, etc. In particular, the secretion of pituitary hormones is regulated by the positive or negative feedback mechanisms, mediated by peripheral hormones secreted from endocrine glands and hypothalamic hormones.

It is known that these hormones and corresponding receptors are not only localized in the hypothalamo-hypophysial system but are also distributed widely in the brain. Further, they are distributed in a similar manner in the peripheral tissues and are thought to have an important function.

For example, the pancreas plays an important role in glucose metabolism by secreting glucagon and insulin as well as digestive juice. Insulin is secreted from β cells of the pancreas, and the secretion is enhanced primarily by glucose. However, various receptors exist on the β cells, and insulin secretion is known to be regulated by a variety of factors other than glucose, including peptide hormones (galanin, somatostatin, gastrin, selectin, gastric inhibitory polypeptide, glucagon, etc.), sugars (mannose, etc.), amino acids, neurotransmitters, and so on.

In the digestive organs, including the stomach, small intestine, and such, food is digested and absorbed by the action of various digestive juices secreted under the control of a number of hormones, hormone-like substances, neurotransmitters, biologically active substances, and so on, including gastrin, selectin, glucagon, gastrin-releasing peptide, vasoactive small-intestinal peptide, acetylcholine, noradrenaline, serotonin, etc. Secretion of these substances is considered to be regulated by their corresponding receptors present in the stomach, small intestine, etc.

Additionally, in the cardiovascular and respiratory systems, represented by the heart and lung, contraction and relaxation of cardiac and vascular smooth muscles, control of blood pressure, and such are strictly regulated under the control of neurotransmitters, hormones, biological active substances, etc.

As described above, a variety of receptor proteins for hormones and neurotransmitters are present in the peripheral tissues, as well as in the brain and pituitary gland, and are thought to play important roles in functional regulations in those tissues.

### DISCLOSURE OF INVENTION

The object of the present invention is to provide a novel G protein-coupled receptor protein, DNA encoding the protein, as well as methods for producing the G protein-coupled receptor protein, and methods for using the protein and DNA.

To isolate the DNA encoding a novel G protein-coupled receptor protein, the inventors screened a human fetal brain cDNA library, based on the Biotin-avidin capture method and using a probe specific for the transmembrane domain of G protein-coupled receptor proteins. As a result, the inventors obtained a novel cDNA fragment encoding a protein with an amino acid sequence that exhibits partial homology with those sequences of known G protein-coupled receptor proteins. The cDNA fragment thus obtained was further used to prepare a probe, which was then used to screen a human heart cDNA library. Consequently, the inventors succeeded in isolating the cDNA that contained a full-length translation frame (open reading frame) (hereinafter referred to as “BG3”).

Analysis of the expression of this gene, by Northern hybridization using RNAs derived from human tissues, revealed that “BG3” is expressed in various tissues; in particular, strong expression was observed in the heart, placenta and lung. Southern hybridization, using human “BG3” as the probe, led to the detection of signals in genomic DNAs from comprehensive species of mammals, suggesting that the “BG3” gene is highly conserved among mammals and has an important function.

The novel G protein-coupled receptor protein, “BG3”, is a relatively large protein compared to other known G protein-coupled receptor proteins, and shows an identity of 24 to 30% and a similarity of 38 to 50% in the transmembrane domain of the protein to the receptor family group classified into Class B receptors, represented by calcitonin receptors, parathyroid hormone (PTH) receptors, glucagon receptors, etc. On the other hand, it shows no homology with any known proteins at its N-terminal extracellular domain. The α-Latrotoxin receptor mentioned above is one of the proteins that shows relatively high amino acid sequence homology with the “BG3” protein of the present invention. The “BG3” receptor protein found by the inventors is thought to be a receptor related to hormone secretion, widely involved in intracorporeal regulation including neurotransmitter secretion.

The “BG3” protein may be advantageously used to screen for its ligands, as well as to screen for candidate pharmaceutical compounds capable of regulating signal transduction from this protein, by utilizing the binding activity or the cell stimulatory activity as an index.

Consequently, the present invention relates to a novel G protein-coupled receptor protein, and the DNA encoding the protein, as well as to methods of screening for candidate ligands and pharmaceutical compounds using the same. More specifically, the present invention provides the following:

(1) DNA encoding a guanosine triphosphate binding protein-coupled receptor protein, wherein said DNA is selected from one of the following:

(a) a DNA encoding a protein consisting of the amino acid sequence of SEQ ID NO: 6;

(b) a DNA comprising the coding region of the nucleotide sequence of SEQ ID NO: 5;

(c) a DNA encoding a protein consisting of the amino acid sequence of SEQ ID NO: 6 in which one or more amino acids are substituted, deleted, inserted and/or added; and

(d) a DNA hybridizing under stringent conditions with a DNA consisting of the nucleotide sequence of SEQ ID NO: 5;

(2) a DNA encoding a partial peptide of the guanosine triphosphate binding protein-coupled receptor protein consisting of the amino acid sequence of SEQ ID NO: 6;

(3) a vector containing the DNA of (1) or (2);

(4) a transformant having the DNA of (1) or (2), or the vector of (3);

(5) a protein or peptide encoded by the DNA of (1) or (2);

(6) a method for producing the protein or peptide of (5), which comprises the steps of culturing the transformant of (4) and collecting the expressed protein from the transformant or culture supernatant thereof;

(7) a method of screening for a ligand that binds to the protein or peptide of (5), wherein the method comprises the steps of:

(a) contacting the test compound with a protein or peptide of (5); and

(b) selecting the compound that binds to said protein or peptide;

(8) a method of screening for a compound that has an inhibitory activity on the binding between the protein or peptide of (5) and the ligand thereof, wherein the method comprises the steps of:

(a) detecting the binding activity of a protein or peptide of (5) to its ligand by contacting said protein or peptide with the ligand in the presence of the test compound; and

(b) selecting a compound that reduces the binding activity of said protein or peptide to the ligand, by comparing the binding activity detected in step (a) with that detected in the absence of the test compound;

(9) a kit for screening for a compound that inhibits the binding of a protein or peptide of (5) to its ligand, wherein said kit comprises the protein or peptide of (5);

(10) an antibody that binds to the protein of (5); and

(11) a DNA hybridizing to the DNA consisting of the nucleotide sequence of SEQ ID NO: 5 or to the complementary strand thereof, wherein said DNA has a length of at least 15 nucleotides.

As used herein, the term “G protein-coupled receptor protein” refers to a receptor protein that mediates intracellular signaling through activation of the G protein. The term “ligand” used herein refers to a naturally occurring compound capable of inducing signal transduction by binding to the G protein-coupled receptor protein. The term “agonist”, as used herein, refers to both compounds that naturally occur and those that are artificially synthesized, wherein an agonist has the same biological activity as the ligand of a G protein-coupled receptor protein. As used herein, the term “antagonist” refers to both compounds that naturally occur and those that are artificially synthesized, wherein an antagonist is capable of inhibiting the biological activity of a ligand of a G protein-coupled receptor protein. In addition, the “protein” and “peptide” of the present invention includes the salt forms thereof.

The present invention provides a novel G protein-coupled receptor protein. The nucleotide sequence of the cDNA of the human G protein-coupled receptor protein “BG3”, which was isolated by the present inventors, is shown in SEQ ID NO: 5, and the amino acid sequence of the “BG3” protein encoded by the cDNA is shown in SEQ ID NO: 6.

The gene encoding the “BG3” protein was isolated from a human fetal brain cDNA library by the biotin-avidin capture method using the Fg probe.

The human “BG3” protein of the present invention contains an open reading frame encoding a protein consisting of 874 amino acid residues. The result of hydrophobicity analysis revealed that the “BG3” protein possesses seven hydrophobic domains characteristic of G protein-coupled receptor proteins (FIG. 1).

The “BG3” protein shares homology with the rat Ca++-independent a-latrotoxin receptor, i.e. showing 38% identity and 56% similarity extending over 328 amino acid residues. The “BG3” protein also shows 27% identity and 49% similarity with several corticotropin releasing factor receptors (CRF-R) over the stretch of 269 amino acid residues.

Expression of the receptor mRNA encoding the “BG3” protein was investigated in the human tissues, and the signals were detected in all the tissues examined (heart, brain, placenta, lung, liver, skeletal muscle, kidney and pancreas). Among these tissues, especially intense signals were detected in heart, placenta and lung.

These facts indicate that the “BG3” protein belongs to the G protein-coupled receptor protein family. Furthermore, the fact that the “BG3” protein is a G protein-coupled receptor protein indicates that this protein mediates signal transduction via the activation of G proteins, particularly by the action of its ligand.

The endogenous ligand for the “BG3” receptor protein has not yet been identified. The protein of the present invention may be used to screen for a ligand useful as a pharmaceutical product or a compound that inhibits the binding of the ligand.

Potential ligands for the protein of the present invention include, but are not limited to, PTH (parathyroid hormone), calcitonin, CGRP (calcitonin gene related peptide), glucagon, selectin, adrenomedullin, serotonin, adrenaline and noradrenaline, galanin, somatostatin, and chemokines.

Aberrations in the signal transduction mediated by the protein of the present invention are thought to result in various diseases. Therefore, compounds that activate the G protein-coupled receptor protein of the present invention or that can inhibit the function of the G protein-coupled receptor protein are expected to be useful as pharmaceuticals. When a compound that activates the G protein-coupled receptor protein of the present invention is used for prophylactic or therapeutic purposes, diseases to be treated may include asthma, Parkinson's disease, acute heart failure, chronic heart failure, congenital heart disease, diabetes, renal failure, cancer, metastatic cancer, hypotension (or hypertension), urine retention, osteoporosis, and so on.

On the other hand, diseases that may be treated with compounds that inhibit activation of the G protein-coupled receptor protein of the present invention for prophylactic and therapeutic purposes may include hypertension (or hypotension), angina pectoris, myocardial infarction, cerebral infarction, diabetes, renal failure, cancer, ulcer, asthma, allergy, benign prostatomegaly, psychopathic and neurological disorders including schizophrenia, manic excitement, depression, delirium, dementia and serious mental retardation, dyskinesia including Huntington's disease and Gilles de la Tourette's syndrome, and so on. Additionally, it is conceivable that a compound that inhibits the G protein-coupled receptor may be beneficial for the reversal of endogenous anorexia and adjustment of pathological starvation.

The protein of the present invention may be prepared as a recombinant protein, by means of recombinant DNA technology, or as a naturally occurring protein. A naturally occurring protein may be prepared, for instance, by performing affinity chromatography on the extract of tissues suspected to express the “BG3” protein (e.g., heart, placenta and lung) with the antibody raised against the “BG3” protein described below. On the other hand, recombinant proteins can be prepared by culturing cells transformed with DNA encoding the “BG3” protein, as described below, then allowing the proteins to be expressed and collecting the expressed proteins.

It is possible for one skilled in the art to prepare an altered protein having a function equivalent to that of the naturally occurring protein (function to mediate intracellular signaling through the activation of the guanosine triphosphate binding protein) by introducing modifications, such as substitution of amino acids, in the naturally occurring human “BG3” protein (the amino acid sequence of “BG3” is shown in SEQ ID NO: 6) according to known methods. Mutations of amino acids in a protein may also occur naturally. Thus, the G protein-coupled receptor proteins of the present invention includes such mutant proteins in which the amino acid sequence is altered from that of the naturally occurring protein by substitution, deletion, addition, insertion, and so on, provided they retain a function equivalent to that of the naturally occurring protein.

Preferably, amino acids are substituted with amino acids of similar nature. For example, Ala, Val, Leu, Ile, Pro, Met, Phe, and Trp are all classified as nonpolar amino acids, and are assumed to share similar nature. Uncharged amino acids include Gly, Ser, Thr, Cys, Tyr, Asn and Gln. Asp and Glu are classified as acidic amino acids. Lys, Arg and His are classified as basic amino acids.

Methods for altering amino acids are known to those skilled in the art and include the Kunkel method (Kunkel T. A. et al., Methods Enzymol. 154:367-382 (1987)), the double primer method (Zoller M. J. and Smith M., Methods Enzymol. 154:329-350 (1987)), cassette mutagenesis (Wells et al., Gene 34:315-323 (1985)), and the megaprimer method (Sarkar G. and Sommer S. S., Biotechniques 8:404-407 (1990)). The number of mutated amino acids in a functionally equivalent protein is generally 10% or less of all the amino acids, preferably 10 amino acids or less, and more preferably 3 amino acids or less (for instance, a single amino acid).

It is also well known within the art that proteins functionally equivalent to the “BG3” protein can be obtained from DNAs homologous to the human “BG3” gene from various organisms, such homologous DNAs being isolated by way of, for example, the hybridization technique (Hanahan, D. and Meselson, M., Meth. Enzymol. 100: 333-342 (1983); Benton, W. D. and Davis, R. W., Science 196: 180-182 (1977)), and such, using the cDNA sequence for human “BG3” (the nucleotide sequence of human “BG3” is shown in SEQ ID NO: 5) or partial sequences thereof. Thus, one skilled in the art may prepare a protein that is functionally equivalent to the human “BG3” protein using DNA that hybridizes with the human “BG3” cDNA. Such proteins are included in the G protein-coupled receptor proteins of the present invention. Animals from which functionally equivalent proteins can be isolated include, but are not limited to, mice, rats, rabbits, cattle, dogs, monkeys, and so on. Tissues such as heart, placenta and lung from such animals are considered suitable for the isolation of cDNAs encoding a protein functionally equivalent to the “BG3” protein.

In general, DNAs encoding a protein functionally equivalent to the human “BG3” protein share high homology with the cDNA sequence of human “BG3” (SEQ ID NO: 5). The term “high homology” refers to at least more than 70%, preferably at least more than 80%, and more preferably at least more than 90% sequence homology at the nucleotide sequence level. Sequence homology may be determined by using the FASTA program.

Stringent hybridization conditions for the isolation of DNAs showing high homology with the human “BG3” cDNA are usually conducted by performing hybridization with “6× SSC, 40% formamide, 25° C.” and washing with “1× SSC, 55° C.”. A preferable condition is “6× SSC, 40% formamide, 37° C.” for hybridization and “0.2× SSC, 55° C.” for washing, and more preferably, “6× SSC, 50% formamide, 37° C.” for hybridization and “0.1× SSC, 62° C.” for washing. Hybridization conditions with the same stringency as those described above may be achieved by one skilled in the art by suitably selecting conditions such as dilution degree of SSC, formamide concentration, temperature, and such.

As used herein, an “isolated nucleic acid” is a nucleic acid, the structure of which is not identical to that of any naturally occurring nucleic acid or to that of any fragment of a naturally occurring genomic nucleic acid spanning more than three genes. The term therefore covers, for example, (a) a DNA which has the sequence of part of a naturally occurring genomic DNA molecule but is not flanked by both of the coding sequences that flank that part of the molecule in the genome of the organism in which it naturally occurs; (b) a nucleic acid incorporated into a vector or into the genomic DNA of a prokaryote or eukaryote in a manner such that the resulting molecule is not identical to any naturally occurring vector or genomic DNA; (c) a separate molecule such as a cDNA, a genomic fragment, a fragment produced by polymerase chain reaction (PCR), or a restriction fragment; and (d) a recombinant nucleotide sequence that is part of a hybrid gene, i.e., a gene encoding a fusion protein. Specifically excluded from this definition are nucleic acids present in mixtures of different DNA molecules, transfected cells, or cell clones, e.g., as these occur in a DNA library such as a cDNA or genomic DNA library.

The term “substantially pure” as used herein in reference to a given polypeptide means that the polypeptide is substantially free from other biological macromolecules. For example, the substantially pure polypeptide is at least 75%, 80, 85, 95, or 99% pure by dry weight. Purity can be measured by any appropriate standard method known in the art, for example, by column chromatography, polyacrylamide gel electrophoresis, or HPLC analysis The invention also includes a polypeptide, or fragment thereof, that differs from the corresponding sequence shown as SEQ ID NO:6. The differences are, preferably, differences or changes at a non-essential residue or a conservative substitution. In one embodiment, the polypeptide includes an amino acid sequence at least about 60% identical to a sequence shown as SEQ ID NO:6, or a fragment thereof. Preferably, the polypeptide is at least 65%, 70%, 75%, 80%, 85%, 90%, 95%, 98%, 99% or more identical to SEQ ID NO:6 and has at least one G-protein coupled receptor protein function or activity described herein. Preferred polypeptide fragments of the invention are at least 10%, preferably at least 20%, 30%, 40%, 50%, 60%, 70%, or more, of the length of the sequence shown as SEQ ID NO:6 and have at least one G-protein coupled receptor protein activity described herein. Or alternatively, the fragment can be merely an immunogenic fragment.

As used herein, “% identity” of two amino acid sequences, or of two nucleic acid sequences, is determined using the algorithm of Karlin and Altschul (PNAS USA 87:2264-2268, 1990), modified as in Karlin and Altschul, PNAS USA 90:5873-5877, 1993). Such an algorithm is incorporated into the NBLAST and XBLAST programs of Altschul et al. (J. Mol. Biol. 215:403-410, 1990). BLAST nucleotide searches are performed with the NBLAST program, score=100, wordlength=12. BLAST protein searches are performed with the XBLAST program, score=50, wordlength=3. To obtain gapped alignment for comparison purposes GappedBLAST is utilized as described in Altschul et al (Nucleic Acids Res. 25:3389-3402, 1997). When utilizing BLAST and GappedBLAST programs the default parameters of the respective programs (e.g., XBLAST and NBLAST) are used to obtain nucleotide sequences homologous to a nucleic acid molecule of the invention.

The present invention also includes a partial peptide of the G protein-coupled receptor proteins described above. The partial peptides of the present invention include, for instance, a peptide corresponding to the binding site, which serves as a binding site for ligands existing in the organisms, of the protein of the present invention. Administration of such partial peptides to the living body enables competitive inhibition of the binding between the receptor protein of the present invention and its ligand, due to the antagonistic binding of the partial peptide to the native ligand. Likewise, the use of partial peptides corresponding to the binding site for the G protein makes it possible to competitively inhibit the binding of the protein of the present invention to the G protein in the cell. These partial peptides are useful as inhibitors of signal transduction mediated by the proteins of the present invention. The partial peptides of the present invention include, for example, the G protein-coupled receptor protein that has its signal sequence removed. Furthermore, the partial peptides of the present invention include, for instance, a partial peptide corresponding to the N-terminal region of the G protein-coupled receptor protein of the present invention, which can be utilized to prepare antibodies. A partial polypeptide containing the amino acid sequence specific for the protein of the present invention has a chain length of at least 7 or 8 amino acid, preferably, at least 15 amino acids, and more preferably, at least 20 amino acids.

The present invention also provides DNAs encoding the G protein-coupled receptor protein of the invention as described above, or partial peptides thereof. The DNAs encoding the G protein-coupled receptor protein of the present invention or partial peptides thereof include, but are not limited to, cDNAs, genomic DNAs, and synthetic DNAs. DNAs having any analogous nucleotide sequence, based on degeneracy of genetic code, are also included, so long as the degenerate DNA encodes a protein of the present invention.

cDNAs encoding the G protein-coupled receptor protein of the present invention can be screened, for example, by labeling the cDNA of SEQ ID NO: 5 or a fragment thereof, RNAs complementary thereto, or synthetic oligonucleotides containing a part of the cDNA sequence, with 32P or the like, then hybridizing them to cDNA libraries derived from tissues expressing the G protein-coupled receptor protein of the present invention (e.g., tissues of heart, placenta and lung). Alternatively, cDNAs may be cloned by synthesizing oligonucleotides corresponding to the nucleotide sequence of the cDNA, and by amplifying the template cDNA from an appropriate tissue (such as tissues from heart, placenta and lung) by PCR. The genomic DNAs can be screened, for example, by labeling the cDNA of SEQ ID NO: 5 or a fragment thereof, RNAs complementary thereto, or synthetic oligonucleotides containing a part of the cDNA sequence, with 32P or the like, and then hybridizing to a genomic DNA library. Alternatively, genomic DNAs may be cloned by synthesizing oligonucleotides corresponding to the nucleotide sequence of the cDNA, and by amplifying the genome DNA as the template by PCR. Synthetic DNAs can be prepared, for example, by chemically synthesizing oligonucleotides comprising part of the cDNA sequence of SEQ ID NO: 5, annealing them into a double strand, and then ligating them using a DNA ligase (Khorana H. G. et al., J. Biol. Chem. 251:565-570 (1976); Goeddel D. V. et al., Proc. Natl. Acad. Sci. USA 76:106-110 (1979)).

These DNA molecules are useful for producing recombinant proteins. That is, the G protein-coupled receptor protein of the present invention may be prepared as a recombinant protein by inserting a DNA encoding the G protein-coupled receptor protein of the present invention (e.g., the DNA of SEQ ID NO: 5) into an appropriate expression vector, culturing a transformant obtained by introducing the vector into an appropriate cell, and purifying the expressed protein. Since the G protein-coupled receptor protein of the present invention is a receptor protein, it can be expressed on the cell membrane for the preparation.

Specifically, if the host is Escherichia coli, plasmid vectors, such as pET-3 (Rosenberg A. H. et al., Gene 56:125-135 (1987)) and pGEX-1 (Smith D. B. and Johnson K. S., Gene 67:31-40 (1988)), may be used. The Hanahan method (Hanahan D., J. Mol. Biol. 166:557-580 (1983)), electroporation (Dower W. J. et al., Nucleic Acids Res. 16:6127-6145 (1988)), and the like may be used for transformation of E. coli. If the host is a fission yeast (Schizosaccharomyces pombe), plasmid vectors such as pESP-1 (Lu Q. et al., Gene 200: 135-144 (1997)), and so on may be used. Yeasts can be transformed, for example, by the spheroplast transformation method (Beach D. and Nurse P., Nature 290:140 (1981)), and the lithium acetate method (Okazaki K. et al., Nucleic Acids Res. 18:6485-6489 (1990)), etc.

If the host is a mammalian cell, such as Chinese Hamster ovary-derived CHO cells and human HeLa cells, vectors such as pMSG (Clontech) can be used. The recombinant DNA may be introduced into the mammalian cell by the calcium phosphate method (Graham F. L. and van derEb A. J., Virology 52:456-467 (1973)), the DEAE-dextran method (Sussman D. J. and Milman G., Mol. Cell. Biol. 4:1641-1643 (1984)), lipofection (Feigner P. L. et al., Proc. Natl. Acad. Sci. USA 84:7413-7417 (1987)), electroporation (Neumann E. et al., EMBO J. 1:841-845 (1982)), or the like.

If the host is an insect cell, baculovirus vectors such as pBacPAK8/9 (Clontech) can be used. Transformation of the insect cell can be performed according to methods known in the art, for example as described in the literature (Bio/Technology 6:47-55 (1980)).

Recombinant proteins expressed in the host cells can be purified by known methods. The proteins can also be synthesized as fusion proteins, for example, histidine residues tagged to the N-terminus, fused to glutathione-S-transferase (GST), and such, and may be purified by binding with a metal chelating resin, or a GST affinity resin (Smith M. C. et al., J. Biol. Chem. 263:7211-7215 (1988)), respectively. For instance, when pESP-1 is used as the vector, the protein of interest is synthesized as a fusion protein with the GST; thus, the recombinant protein can be purified using a GST affinity resin. The fusion protein may be cleaved with thrombin, blood coagulation factor Xa, and such to liberate the protein of interest from the fusion protein.

In addition, the DNA encoding the G protein-coupled receptor protein of the present invention is applicable to gene therapy of diseases associated with mutations in the inventive protein. When used in gene therapy, the DNA is introduced into human cells by conventional methods, such as those utilizing retrovirus vectors (Danos O. and Mulligan R. C., Proc. Natl. Acad. Sci. USA 85:6460-6464 (1988); Dranoffet al., Proc. Natl. Acad. Sci. USA 90:3539-3543 (1993)), adenovirus vectors (Wickham T. J. et al., Cell 73:309-319 (1993)), etc. To administer the vector to patients, transplantation of bone marrow, subcutaneous injection, and intravenous injection can be used (Asano S., Protein Nucleic acid and Enzyme 40:2491-2495 (1995)).

The present invention also provides DNAs comprising the nucleotide sequence of SEQ ID NO: 5, or those hybridizing specifically to the complementary strand thereof, which have a chain length of at least 15 nucleotides. The DNA is preferably a DNA consisting of the nucleotide sequence of SEQ ID NO: 5, or those that hybridize specifically to the complementary strand thereof. As used herein, the term “hybridize specifically” means that no significant cross-hybridization occurs with DNAs encoding other proteins under ordinary conditions, preferably under stringent conditions for hybridization. The conditions described above can be used for the hybridization.

These DNAs of the present invention include DNAs encoding the protein of the present invention, and probes, primers, nucleotides and nucleotide derivatives (e.g., antisense oligonucleotides, ribozyme, etc.) which are capable of hybridizing specifically to a complementary DNA of the DNA.

Accordingly, in one aspect, the invention provides an isolated or purified nucleic acid molecule that encodes a polypeptide described herein or a fragment thereof. Preferably, the isolated nucleic acid molecule includes a nucleotide sequence that is at least 60% identical to the nucleotide sequence shown in SEQ ID NO:5. More preferably, the isolated nucleic acid molecule is at least 65%, 70%, 75%, 80%, 85%, 90%, 91%, 92%, 93%, 94%, 95%, 96%, 97%, 98%, 99%, or more, identical to the nucleotide sequence shown in SEQ ID NO:5. In the case of an isolated nucleic acid molecule which is longer than or equivalent in length to the reference sequence, e.g., SEQ ID NO:5, the comparison is made with the full length of the reference sequence. Where the isolated nucleic acid molecule is shorter that the reference sequence, e.g., shorter than SEQ ID NO:5, the comparison is made to a segment of the reference sequence of the same length (excluding any loop required by the homology calculation).

The cDNAs as described above, that encode the G protein-coupled receptor protein of the present invention or oligonucleotides comprising a part thereof, can be used to clone the gene or cDNA, or for amplification of such by PCR. Moreover, polymorphism or abnormality of the gene and cDNA can be detected by methods such as restriction fragment length polymorphism (RFLP), and single-stranded conformational polymorphism (SSCP).

The present invention further provides antibodies that bind to the G protein-coupled receptor protein of the present invention. The antibody that binds to the G protein-coupled receptor protein of the present invention can be prepared by using methods known in the art (for instance, see Shin-Seikagaku-Jikken-Kouza (New Course on Biochemical Experiments) I: Protein I 389-406, Tokyo-Kagaku-Doujin).

For instance, polyclonal antibodies can be prepared by the procedure as follows: An appropriate dose of the proteins or peptides above is administered to an animal, such as rabbit, guinea pig, mouse, chicken and such for immunization. The proteins or peptides may be administered together with an adjuvant (such as FIA or FCA) that promotes antibody production. Administration is usually carried out every few weeks. The titer of antibodies can be increased by multiple immunizations. After the final immunization, antisera are obtained by withdrawing blood from the immunized animal. Polyclonal antibodies can be purified from antisera by, for example, performing ammonium sulfate precipitation, fractionation using anion exchange chromatography, or affinity chromatography using Protein A or immobilized antigen.

On the other hand, monoclonal antibodies, for example, are prepared as follows: The G protein-coupled receptor protein of the invention or its partial peptide is administered to animal as described above. After the final immunization, the spleen or lymph node is excised. Then, antibody-producing cells are recovered from the spleen or the lymph node, and fused with myeloma cells using, for example, polyethylene glycol and such, to produce hybridomas. Object hybridomas are screened, and then cultured. The monoclonal antibody can be prepared from the culture supernatant. The monoclonal antibody can be purified by, for example, performing ammonium sulfate precipitation, fractionation using anion exchange chromatography, or affinity chromatography using either Protein A or immobilized antigen.

Antibodies thus prepared can be used, for example, in diagnosis and antibody therapy of diseases associated with aberrant expression of the G protein-coupled receptor protein of the present invention and for detection of the expression level of the G protein-coupled receptor protein of the present invention, as well as for affinity purification of the G protein-coupled receptor protein of the invention.

It is preferable to use humanized antibodies or human antibodies for the antibody therapy. The humanized antibody, like a mouse-human chimeric antibody, can be prepared, for example, as follows: (1) isolate the gene encoding the antibody against the G protein-coupled receptor protein of the present invention from antibody-producing mouse cells; (2) replace the constant region of the H chain of the antibody with that of the human IgE; and (3) introduce into mouse myeloma J558L cells (Neuberger M. S. et al., Nature 314:268-270 (1985)). Alternatively, human antibodies can be prepared by immunizing mice whose immune systems have been replaced with that of humans with the G protein-coupled receptor protein of the present invention.

Furthermore, the present invention provides a method of screening for ligands of the G protein-coupled receptor protein of the present invention. The method includes the steps of: (1) exposing a test compound to the G protein-coupled receptor protein of the present invention or to its partial peptide, and (2) selecting compounds that bind to the proteins or the peptides. Compounds to be tested include known compounds such as acetylcholine, adenosine, adrenaline, noradrenaline, angiotensin, bombesin, bradykinin, C5a anaphylatoxin, calcitonin, cannabinoids, chemokines, cholecystokinin, dopamine, endothelin, formylmethionylpeptide, GABA, galanin, glucagon, glutamate, glycopeptide hormone, histamine, 5-hydroxytryptophan, leucotriene, melanocortin, neuropeptide Y, neurotensin, odorant, opioid peptide, opsin, parathyroid hormone, platelet activating factor, prostanoid, somatostatin, tachykinin, thrombin, thyrotropin releasing hormone, vasopressin, oxytocin (Watson S. and Arkinstall S., The G protein Linked Receptor Facts Book, Academic Press (1994)), CGRP (calcitonin gene related protein), adrenomedullin, amylin, and serotonin; other purified proteins; gene (including library) products; extracts of tissues or cells in which the existence of the ligand is predicted (e.g., heart, lung, placenta, etc.); and culture supernatants.

The G protein-coupled receptor protein of the present invention used for the above-described screening may be as expressed intracellularly, or as expressed on the surface of the desired cells (including transformants genetically engineered to express the protein), in the form of membrane fractions of the cells, or bound to an affinity column. Test compounds used for screening may be, if necessary, suitably labeled. Labels includes, but are not limited to, radioisotope labels, fluorescence labels, and so on.

The binding of the G protein-coupled receptor protein of the present invention and a test compound can be detected through the labeling of the test compound (for instance, by measuring the radioactivity or fluorescence intensity), as well as by using, as an index, intracellular signaling mediated by the binding of the compound to the G protein-coupled receptor protein of the present invention present on the cell surface (such as G protein activation, changes in the concentration of Ca²⁺ or cAMP, phospholipase C activation, and changes in pH). The detection can be performed based on specific methods described in the literatures (Cell Calcium 14:663-671 (1993); Analytical Biochemistry 226:349-354 (1995); J. Biol. Chem. 268:5957-5964 (1993); Cell 92:573-585 (1998); Nature 393:272-273 (1998)) or in the unexamined published Japanese patent application (JP-A) No. Hei 9-268. Alternatively, the binding may be detected by measuring the activity of a reporter gene using two-hybrid system (Zervos et al., Cell 72:223-232 (1994); Fritz et al., Nature 376:530-533 (1995)).

The present invention also provides a method of screening for a compound which can inhibit the binding between the G protein-coupled receptor proteins of the invention and their ligands. The method includes the steps of: (a) exposing the ligand to the G protein-coupled receptor proteins of the present invention or their partial peptides in the presence of a test compound, and detecting the binding activity between the proteins or the partial peptides and the ligand, and (b) comparing the binding activity detected in (a) with that in the absence of the test compound, and selecting a compound that reduces the binding activity. Compounds to be tested include proteins, peptides, non-peptide compounds, artificially synthesized compounds, extracts of tissues and cells, sera, and such, but are not limited thereto. The G protein-coupled receptor proteins of the present invention used for screening may be as expressed in desired cells (including transformants genetically engineered to express the proteins) or as expressed on the cell surface, in form of membrane fractions of the cells, or bound to an affinity column. If necessary, ligands for screening may be labeled appropriately. Labels include radioisotopic labels, fluorescent labels, and such, but are not limited thereto.

The binding activity between the G-protein coupled receptor proteins of the present invention, or their partial peptides, and ligands can be examined by detecting the label tagged to the ligand (for instance, by measuring the radioactivity or fluorescence intensity), as well as by using, as an index, signal transduction into the cell(such as G protein activation, changes in the concentration of Ca²⁺ or cAMP, phospholipase C activation, and changes in pH), which are triggered by the compound binding to the G protein-coupled receptor protein of the invention on the surface of the cell. Specific examples of such methods are described in the literatures (See Cell Calcium 14:663-671 (1993); Analytical Biochemistry 226:349-354 (1995); J. Biol. Chem. 268:5957-5964 (1993); Cell 92:573-585 (1998); Nature 393:272-273 (1998)), and JP-A No. Hei 9-268.

As a result of detection, if the binding activity under the presence of a test compound is reduced compared to that in the absence of the compound (control), such a compound is determined to be an inhibitor of the binding between the G protein-coupled receptor protein of the present invention (or its partial peptides) and the ligand. Such compounds include those capable of triggering signaling into the cell through the binding to the G protein-coupled receptor protein of the invention (agonist), and those absent of such activity (antagonist). Agonists present biological activities similar to that of the ligands of the G protein-coupled receptor protein of the present invention. On the other hand, antagonists inhibit the biological activity of the ligands of the G protein-coupled receptor protein of the present invention. Accordingly, these agonists and antagonists may be useful as pharmaceutical compositions for treatment of diseases arising from disorders in the signaling pathway mediated by the G protein-coupled receptor protein of the present invention.

When a compound isolated by the screening method of the present invention is to be used as a drug, the compound may be directly administered to the patient, or it may be formulated by commonly known methods for pharmaceutical preparations. For example, the compound may be formulated by properly combining with, for example, pharmacologically acceptable carriers or media, specifically, sterilized water, physiological saline, vegetable oils, emulsifiers, suspending agents, surfactants, stabilizers, binders, lubricants, edulcorants, spices, coloring agents, and so on. Administration to patients is performed according to the methods commonly known to those skilled in the art, including intranasal, bronchial, intramuscular, and oral administrations, as well as by intra-arterial, intravenous, and subcutaneous injections, and such. Doses will vary depending on factors such as body-weight, age of the patient, and the method for administration. However, one skilled in the art can apropriately select suitable doses. If the compound can be encoded by a DNA, such a DNA may be integrated into a gene therapy vector for use in gene therapy as described above. Doses and administration methods will vary depending on factors such as body-weight, age of the patient, state of the disease, and so on. However, one skilled in the art can appropriately select suitable doses and methods.

Furthermore, the present invention provides a kit to screen for a compound that inhibits binding between the G protein-coupled receptor protein of the present invention and its ligand. One feature of the kit is that it contains the G protein-coupled receptor protein of the present invention or a partial peptide thereof. The G protein-coupled receptor protein of the present invention (or a partial peptide thereof) in the kit of the present invention may take its form as expressed in the desired cells (including transformants genetically engineered to express the protein) or as expressed on the cell surface, or it may be in a form of membrane fractions of the cell, or bound to an affinity column. In addition to a receptor protein sample as described above, the kit of the invention may include a ligand sample (both labeled and unlabeled), buffer for the receptor protein-ligand reaction, washing solution, and so on. Labels of the ligands include, for instance, radioactive labels and fluorescent labels. The kit of the present invention may be used, for example, according to the description of JP-A No. Hei 9-268.

### DESCRIPTION OF DRAWINGS

FIG. 1 shows the hydrophobicity plot of the human “BG3” protein. In the figure, numbers I to VII indicate the seven hydrophobic regions (transmembrane regions) characteristic of G protein-coupled receptor proteins. The numbers on the top indicate the positions of the amino acid residues in the “BG3” protein.

FIG. 2 is an electrophoresis photograph of the results of Northern blot analysis of the tissue specific expression of the human “BG3” gene.

FIG. 3 is an electrophoresis photograph of the results of Southern blot analysis of the human “BG3” gene.

### BEST MODE FOR CARRYING OUT THE INVENTION

The present invention is illustrated in detail below with reference to the examples, but is not to be construed as being limited thereto.

### EXAMPLE 1

### Isolation of the Human G Protein-Coupled Receptor Gene

1. Manipulation of the Gene Trapper and Design of the Probe.

The G protein-coupled receptors are structurally characterized by seven transmembrane regions, and the DNA and amino acid sequences of the transmembrane regions and their adjacent regions are well conserved. Thus, sense primer Fg (5′-BATNGCCAAC CTBKCCTTCT C-3′/SEQ ID NO: 1; 21mer, Degeneracy 72) (B=G, T or C, K=G or T, and N=A, C, G or T) was synthesized on the basis of the comparison of the DNA sequence of the second transmembrane domain conserved among the known G protein-coupled receptors, namely the mouse neuropeptide Y receptor Y1 (GenBank Accession Number Z18280), rat Y1 (Z11504), human Y1 (M84755), mouse neuropeptide Y receptor Y4 (U40189), rat Y4 (Z681 80), human Y4 (Z66526), and mouse neuropeptide Y receptor Y6 (U58367). Mixed nucleotides were used to increase the sequence identity with different receptor cDNAs.Gene Trapper (GIBCO BRL) was used in the Biotin-Avidin Capture method. In this method, the library of plasmids containing the f1 ori sequence was digested to a single-stranded plasmid library, using Gene II and Exo III nuclease. A sense primer for the gene of interest was synthesized and biotinylated to provide a probe. The biotinylated probe was allowed to hybridize with the single-stranded plasmid library, and single-stranded plasmid DNA to which the probe was specifically bound was collected using streptavidin-coated magnetic beads and then washed. Double-stranded plasmid DNA was synthesized again from this single-stranded plasmid DNA by using the same primer. XL1 Blue MRF' cells (host E. coli cells) were transformed with the DNA thus synthesized using E. coli pulser (BIORAD). The procedure was performed according to the details of the protocol provided by the manufacturer. 384 clones were randomly selected from the clones obtained by the Gene Trapper method using a human fetal brain cDNA library (GIBCO BRL) as the screening source, inoculated in the LB medium containing 100 μg/ml of ampicillin and cultured overnight at 37° C. The plasmid DNAs were purified with BioRobot9600 (QIAGEN) using the QIA Prep 96 Turbo BioRobot Kit (QIAGEN). For sequencing, the sequencing reaction was carried out using ABI PRISM877 (ABI) with BigDye Primer Cycle Sequencing Ready Reaction Kit (ABI), followed by electrophoresis using automated fluorescence-based DNA sequencer ABI PRISM 377 DNA Sequencer (ABI). Partial nucleotide sequence at the 5′ terminal region of each clone was determined, which was further analyzed by homology searches (BLAST search) in various databases. As a result, inventors discovered the clone GFgHG0360 (SEQ ID NO: 2), which appeared to encode a novel G protein-coupled receptor that showed significant homology to known G protein-coupled receptors. The novel G protein-coupled receptor predicted to be encoded by the clone was designated “BG3”.

2. Sequencing

The nucleotide sequence of clone GFgHG0360 was determined using the shotgun cloning method (Sambrook et al., Molecular Cloning: A laboratory manual 2^{nd} edition (1989)). Biorupter (Tosyo Denki Co.), a closed ultrasonic homogenizer for biomaterials, was used for fragmentation of the cDNA, and the DNA fragments were fractionated by electrophoresis on a 1.2% agarose gel. The gel slice containing the DNA fragments about 0.5-1.0 kb in length was cut out, and the DNA fragments were purified with Gene Clean (Bio101), blunt-ended with T4 DNA polymerase (TaKaRa), and subcloned. Clones thus obtained were subjected to the sequencing reaction. The DNA sequences obtained were analyzed using the sequencing softwares Sequencher (Hitachi Software Engineering) and RASERGENE (DNASTAR). The results showed that the sequence contained a poly (A) tail at the 3′ terminus but lacked a stop codon upstream of the initiation codon at the 5′-side, suggesting that the sequence does not contain the complete open reading frame.

### EXAMPLE 2

### Northern Blot Analysis of Human BG3

To study the expression pattern of the receptor mRNA encoding the “BG3” protein expressed in human tissues, Northern blot analysis was performed using human MTN Blot I (CLONTECH).

Based on the previously determined nucleotide sequence of clone GFgHG0360, sense and antisense primers having the following sequences were synthesized:

BG3F01: 5′-CCACGCCAACCTGTCCTTCGC-3′/SEQ ID NO: 3
and
BG3R04: 5′-CTGCTCGTGAGCGACCAGACC-3′/SEQ ID NO: 4.

Conditions for the PCR were: 30 cycles of 94° C. for 240 seconds, 94° C. for 10 seconds, 55° C. for 5 seconds, and 74° C. for 60 seconds. PCR was performed using a reaction mixture containing 1× Buffer (standard buffer supplied with the kit), 0.2 mM dNTPs, 1 mM of each primer, 5 ng of template DNA (GFgHG0360), and 0.25 Unit/ml KOD' polymerase (TOYOBO). The DNA band, having a size of about 0.7 kb, was cut out from the agarose gel after electrophoresis and purified with Gene Clean (Bio 101) to provide the template for preparing the probes.

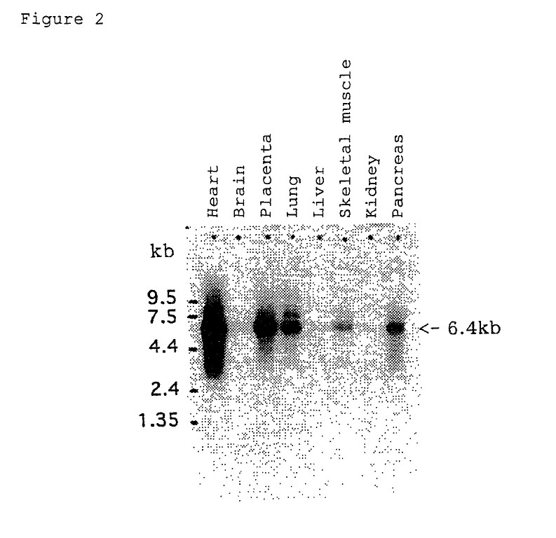

The probe was prepared by labeling with [α-³²P]dCTP (Amersham) according to the protocol supplied with the Prime-It II Random Primer Labeling Kit (Stratagene). Conditions for hybridization and washing were determined according to the manufacturers' protocol provided with the Human MTN Blot I (CLONTECH). Final washing was done with 0.1× SSC-0.1% SDS for 30 minutes at 55° C. Northern blot analysis revealed signals in all the tissues tested (heart, brain, placenta, lung, liver, skeletal muscle, kidney and pancreas). Among them, particularly intense signal was detected in heart, placenta and lung (FIG. 2).

### EXAMPLE 3

### Isolation of the Full-Length Clone of Human “BG3”

To efficiently screen for the full-length cDNA clone of human “BG3”, the ZAP II cDNA library (Stratagene) derived from human heart, whose abundant expression of human “BG3” mRNA was confirmed by the Northern blot analysis, was used for the screening. The same human “BG3” as the Northern blot analysis, was used for the screening, and 24 positive clones were obtained. A single cDNA clone having an insert of 5.4 kb that was predicted to encode the 5′-upstream region, the complete open reading frame and the 3′-downstream region of “BG3” was obtained as the result of the 5′- and 3′-sequencing of the clones.

This clone (“E. coli hBG3-17”) was deposited in the National Institute of Bioscience and Human-Technology, Agency of Industrial Science and Technology.

Name of the depositary institution:

The National Institute of Bioscience and Human-Technology, Agency of Industrial Science and Technology, Ministry of International Trade and Industry.

Address of the depositary institution:

1-1-3 Higashi, Tsukuba, Ibaraki 305-8566, Japan.

Date of deposit (original deposit): March 15, 1999.

Accession Number: FERM BP-7070

The partial nucleotide sequences of the 5′ region of GFgHG0360 and the 24 clones, which were obtained by the screening of the human heart ZAP II cDNA library, were aligned to synthesize the primers for sequencing. BigDye Terminator Cycle Sequencing Ready Reaction Kit (ABI) was used to determine the nucleotide sequence of the human “BG3” clone (E. coli hBG3-17). The DNA sequence determined was analyzed using DNA sequencing softwares Sequencher (Hitachi Software Engineering) and RASERGENE (DNASTAR).

The result revealed that the cDNA (SEQ ID NO: 5) contained a translation frame (open reading frame) encoding a protein consisting of 874 amino acid residues (SEQ ID NO: 6). This cDNA appeared to contain the complete ORF, since it comprised a poly(A) tail at the 3′ end and a termination codon upstream of the initiation codon at the 5′ terminal region.

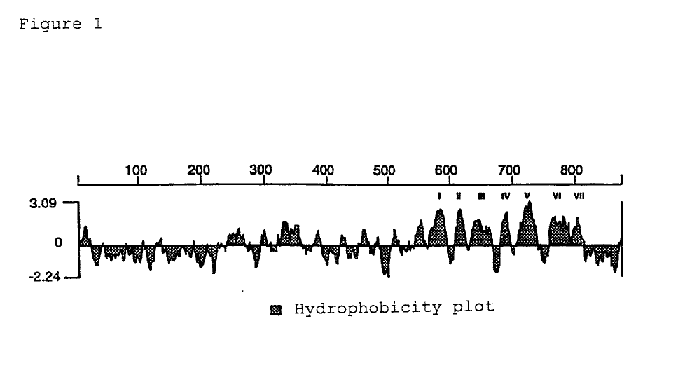

According to the analysis of the amino acid sequence of this DNA, seven transmembrane regions, from the first to the seventh, in the hydrophobic domain characteristic for the G protein-coupled receptor proteins were identified on the hydrophobicity plots (FIG. 1). Further, a signal peptide rich in hydrophobic residues was also identified at the N-terminus.

### EXAMPLE 4

### Southern Blot Analysis of “BG3”

Analysis was performed to determine whether genes encoding the proteins functionally equivalent to the “BG3” protein were conserved among species. ZOO-BLOT (CLONTECH), on which genomic DNA from human, monkey, rat, mouse, dog, cattle, rabbit, chicken and yeast completely digested with endonuclease EcoRI were blotted, was used for the Southern blot analysis.

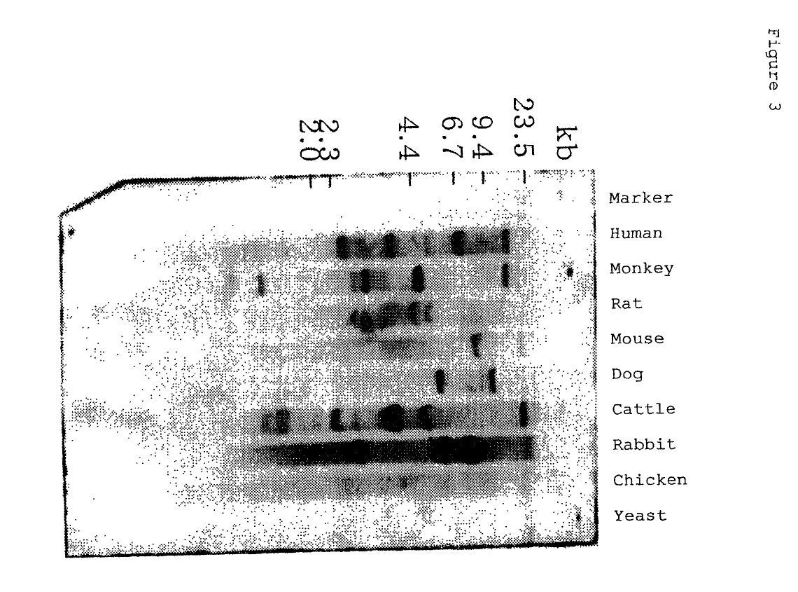

A probe labeled with ³²P was prepared as described above in Example 2, using the same primers that were used in the Northern blot analysis of “BG3”. Hybridization and washing were carried out according to the protocol of ZOO-BLOT (CLONTECH). Final washing was performed in 0.1× SSC-0.1% SDS for 30 minutes at 55° C. Multiple band signals were detected in all the mammals tested including human, monkey, rat, mouse, dog, cattle, and rabbit by the Southern blot analysis, which was performed against genomic DNAs of human, monkey, rat, mouse, dog, cattle, rabbit, chicken, and yeast (FIG. 3).

The fact that signals were detected in the genomic DNA from mammals by the hybridization using the DNA encoding the human “BG3” protein as a probe indicated that a gene encoding the protein functionally equivalent to the “BG3” protein was conserved among species. This further suggested that the “BG3” protein played an important role and was a potential target protein in developing therapeutic agents for various diseases.

The result, that multiple bands were detected by the Southern blot analysis, suggested that the DNA encoding the protein with equivalent function as the “BG3” protein have a structure consisting of multiple exons and introns on the chromosome. Such structure may allow synthesis of different splicing forms of the “BG3” protein from a single gene encoding the “BG3” protein by way of various alternative splicing. Alternatively, it may suggest the existence of genes encoding other proteins that have functions equivalent to the “BG3” protein, or genes encoding different proteins having partial homology to the “BG3” protein but whose function is different from that of the “BG3” protein.

All references and patents cited herein are incorporated by reference in their entirety.

### INDUSTRIAL APPLICABILITY

The present invention provides a novel G protein-coupled receptor protein which is expressed in various tissues, as well as the DNA encoding the protein. Use of the inventive receptor protein makes it possible to screen for ligands and candidate medicinal compounds. It is expected that these ligands and candidate compounds may be used, for example, in the diagnosis, treatment, and such of diseases associated with disorders in the signal transduction system mediated by the G protein-coupled receptor protein of the invention.

What is claimed:

1. A substantially pure polypeptide comprising an amino acid sequence at least 70% identical to SEQ ID NO:6, wherein the polypeptide has a G protein-coupled receptor protein activity.

2. The polypeptide of claim 1, wherein the amino acid sequence is at least 80% identical to SEQ ID NO:6.

3. The polypeptide of claim 1, wherein the amino acid sequence is at least 90% identical to SEQ ID NO:6.

4. A substantially pure polypeptide, the sequence of which consists of SEQ ID NO:6.

5. A substantially pure polypeptide, the sequence of which consists of SEQ ID NO:6 with up to 30 conservative amino acid substitutions, deletions or insertions, wherein the polypeptide has a G protein-coupled receptor protein activity.

6. A substantially pure polypeptide comprising the sequence of SEQ ID NO:6, or a fragment thereof that (a) has a G-protein receptor coupled protein activity or (b) is immunogenic.

7. A substantially pure polypeptide encoded by a nucleic acid that hybridizes under high stringency conditions to the sequence of SEQ ID NO:5, wherein the polypeptide has a G protein-coupled receptor protein activity.

8. An isolated nucleic acid encoding the polypeptide of claim 1.

9. An isolated nucleic acid encoding t he polypeptide of claim 4.

10. An isolated nucleic acid encoding the polypeptide of claim 5.

11. An isolated nucleic acid comprising a strand that hybridizes under high stringency conditions to the sequence of SEQ ID NO:5, or the complement of SEQ ID NO:5.

12. The isolated nucleic acid of claim 11, wherein the nucleic acid encodes a polypeptide having a G protein-coupled receptor protein activity.

13. The nucleic acid of claim 11, wherein the strand is at least 15 nucleotides in length.

14. An isolated nucleic acid comprising the sequence of SEQ ID NO:5.

15. An isolated nucleic acid comprising a sequence encoding the polypeptide of SEQ ID NO:6.

16. An antibody that specifically binds to the polypeptide consisting of SEQ ID NO:6.

17. A vector comprising the nucleic acid of claim 8.

18. A vector comprising the nucleic acid of claim 11.

19. A vector comprising the nucleic acid of claim 14.

20. A vector comprising the nucleic acid of claim 15.

21. A cultured host cell comprising the nucleic acid of claim 8.

22. A cultured host cell comprising the nucleic acid of claim 11.

23. A cultured host cell comprising the nucleic acid of claim 14.

24. A cultured host cell comprising the nucleic acid of claim 15.

25. An antibody that specifically binds to the polypeptide of claim 1.

26. A method of producing a polypeptide, the method comprising culturing the cultured host cell of claim 21 under conditions that permit expression of the polypeptide in the cell.

27. A method for identifying a compound that modulates a G protein-coupled receptor activity, comprising the steps of:
a) contacting a polypeptide of claim 1, or a cell transfected with a nucleic acid encoding the polypeptide of claim 1, with a test compound; and
b) determining whether the test compound modulates a G protein-coupled receptor activity of the polypeptide or cell, thereby identifying a compound that modulates a G protein-coupled receptor activity.

28. A kit comprising the polypeptide of claim 1 and instructions for use in a method of screening.

29. A compound isolated by the method of claim 27.

30. A pharmaceutical composition comprising the compound of claim 29 as an active ingredient.


SEQUENCE LISTING (6 sequences)

SEQ ID NO: 1 - DNA, 21 bp, Artificial Sequence
  Artificially synthesized primer sequence
batngccaacctbkccttctc

SEQ ID NO: 2 - DNA, 3117 bp, Homo sapiens
atcagctatgtgggctgctccctctccgtgctctgcctggtggccacgctggtcaccttcgccgtgctgtcctccgtgagcaccatccggaaccagcgctaccacatccacgccaacctgtccttcgccgtgctggtggcccaggtcctgctgctcattagtttccgcctcgagccgggcacgaccccctgccaagtgatggccgtgctcctacactacttcttcctgagtgccttcgcatggatgctggtggaggggctgcacctctacagcatggtgatcaaggtctttgggtcggaggacagcaagcaccgttactactatgggatgggatggggttttcctcttctgatctgcatcatttcactgtcatttgccatggacagttacggaacaagcaacaattgctggctgtcgttggcgagtggcgccatctgggcctttgtagcccctgccctgtttgtcatcgtggtcaacattggcatcctcatcgctgtgaccagagtcatctcacagatcagcgccgacaactacaagatccatggagaccccagtgccttcaagttgacggccaaggcagtggccgtgctgctgcccatcctgggtacctcgtgggtctttggcgtgcttgctgtcaacggttgtgctgtggttttccagtacatgtttgccacgctcaactccctgcagggactgttcatattcctctttcattgtctcctgaattcagaggtgagagccgccttcaagcacaaaaccaaggtctggtcgctcacgagcagctccgcccgcacctccaacgcgaagcccttccactcggacctcatgaatgggacccggccaggcatggcctccaccaagctcagcccttgggacaagagcagccactctgcccaccgcgtcgacctgtcagccgtgtgagccgggaggctgccaaccaggccaggctgcgctcagaacacacccccccaaacagaatgaaatgccccacctttgcccatggaccctctccttgctgctgtctggacatgggtgttgtggccccgagacagctgtcctcccctgtgactctggctgtcggagcacactgctcagcccagcagcctgatgcccaggccagcgtgggccctcctgccttgcatccacccgtgggctgagtgacttcctcgggggattcccaggacacagtggcctgactgtgatggtgcccttgagcctcccttcatcactcagcatcagacccagcgaggccaggacactcggggccggtcccgcagcaccaggaggggatgttcagcctctgtgccttggtggggcttggggactcagggccaaagaggtggttcaggtccccacgcaccctcagtcaggcgcaggcagctgggggtgtgtggggaagagcatgcggagtccccagtgtctgaatccactgagtggtgagttccccacagccggcgctagctggtgtgtgtctctgtaggtggtgccggcgtgggccaacctgtgctgtgtcatcagttgggggcccctgcccaagccgagctcgagccgtgggcgggagtcgttgactctccaggtgagggcgacccctctgccctgtccttgggggggtcccctctgctcacgtgaagagccgctctgggccttgaggctgcctgatggtgcctgtgcttgggggagcttctcggccatccgctgtgagttttgcctctttggaccccaattcggccttaagatgccctcctccctcgtgtgccagcctccttggttgttcttgggccacaggagctggccgtgtccccgcagtgcctggtgtccaggtggaaagtggagggcattttccagggcactgctttccccagaggcttcctcatggctcacaggcactctacgaagtttctaatgggcagaccacgcggcaggtagcacagtgcgctccgtctggtcaccatgagaccgacctgcgctgagtccccactgacctggagagggagggctggtgacagccgtgtcttctgtgttgagggaaatttatggactcagattcagccccagaggagatgggataattgttatggacccatgtgtgggcatgatcctgtggaacacaggtttgggatcatagatgtgaattaagacaccaccgagatacgggctgtgaggttcatactgtgctgatagcactcgtggtgtctgtgaaatgtgggtaagacattcaaacctggttttgatactggaaactcttcctttaaaactgtgaccatgatttcattcagcccctccacacccctatgtctgccttgtttcagagtgagttttctatggagcctgtggcccttttgcagcccacctggtggcttcttaatgtaactcttcccctggtcgcctggagtggaccactcatctgcaggcctctcctgcatggggagggtaggcagggagcagcatgtctgcaggggtgaacctttgctcttctgtcaggcgaggcccaggctgcaccagccacctgccacatggtgacagtgccacgggccctgcgtatggcccctgcaaccgtgctctggcgggcacacctggctgctgcaggccaaggccgctgttcagtgaagagtcccatgtttagtatggactaaagtcccatgtttagccactgccccaggctcccgtgaccccagaaaccaggtcacatggaccacagtgccagatcctcatcacgccggtgagcacctagaagtgagaacactgtattcctacaatgtacacttggatatttctccttatttagtttctagtgaaacaaatcaagtaaggaactatctttagtttagatggaattatttgtttttaattgttgccgtattcatctatatagctaatatttcaagataagtaatgaacaaaacctgtctaaaccttttgtttccaatgaatgaaagtcatgcactttatttataggctctatgttttggcttctgcagtacttttattatctatacataatttggccaaaaataagaaattggaaagaatgaaatgtttagtttatagtagaagaaagatgatgacactaagttgtgaaaatatgttgtgatttttatgaaataaactcatgtcctg

SEQ ID NO: 3 - DNA, 21 bp, Artificial Sequence
  Artificially synthesized primer sequence
ccacgccaacctgtccttcgc

SEQ ID NO: 4 - DNA, 21 bp, Artificial Sequence
  Artificially synthesized primer sequence
ctgctcgtgagcgaccagacc

SEQ ID NO: 5 - DNA, 5340 bp, Homo sapiens
  CDS (517)...(3138)
ttgagccagaccagaaggagctcgagagcggccgcaggacaagcccgaggagcaggcgggcgctccaggggaaaaccacgcacaaaaccttcttcagagaaaagggaagctccaaacctgactgagacaaacggaggctcttgaaataaaaagaaaataccgcaggacaaacagcctcccgtccccgggcgcaggtcgcggtcacagtggtgacctgggattgctttcccaggactgcgagtcgggtttgggtttctcctccctgcattccacagctgctctggtcatcgcaacgtgtttattgatcactgaagaatctcaagttttgagacgaggaagaaacacccattaggtctccaagacagctgtgtttcacaaactttagggagacagaaattttctcccctggaacctgtgaaaatgtcccttttccaaggaagtgaaggttaagaggtcccgttctcacagaccctcagtaatttcacttggctccgagctttgacctccgagagagccatggaaaagctgctgcggctgtgctgctggtactcctggctgctgctattttattacaactttcaggtgcgtggcgtctactccagatcgcaggaccatccaggatttcaggtgttggcgtctgcttcccattactggccactggagaatgtggatgggatccatgaacttcaggatacaactggagatattgtggaagggaaggtcaacaaaggcatttacctgaaagaggaaaagggagtcacgcttctctattacggcaggtacaacagctcctgcatcagcaagccagagcagtgtggccctgaaggggtcacgttttcttttttctggaagacacaaggagaacagtctagaccaatcccttctgcgtatgggggacaggtcatctccaatgggttcaaagtctgctccagcggtggcagaggctctgtggagctgtacacgcgggacaattccatgacatgggaggcctccttcagccccccaggcccctattggactcatgtcctatttacatggaaatccaaggagggcctgaaagtctacgtcaacgggaccctgagcacctctgatccgagtggaaaagtgtctcgtgactatggagagtccaacgtcaacctcgtgatagggtctgagcaggaccaggccaagtgttatgagaacggtgctttcgatgagttcatcatctgggagcgggctctgactccggatgagatcgccatgtacttcactgctgccattggaaagcatgctttattgtcttcaacgctgccaagcctcttcatgacatccacagcaagccccgtgatgcccacagatgcctaccatcccatcataaccaacctgacagaagagagaaaaaccttccaaagtcccggagtgatactgagttacctccaaaatgtatccctcagcttacccagtaagtccctctcggagcagacagccttgaatctcaccaagaccttcttaaaagccgtgggagagatccttctactgcctggttggattgctctgtcagaggacagcgccgtggtactgagtctcatcgacactattgacaccgtcatgggccatgtatcctccaacctgcacggcagcacgccccaggtcaccgtggagggctcctctgccatggcagagttttccgtggccaaaatcctgcccaagaccgtgaattcctcccattaccgcttcccggcccacgggcagagcttcatccagatcccccacgaggccttccacaggcacgcctggagcaccgtcgtgggtctgctgtaccacagcatgcactactacctgaacaacatctggcccgcccacaccaagatcgcggaggccatgcatcaccaggactgcctgctgttcgccaccagccacctgatttccctggaggtgtccccaccacccaccctgtctcagaacctgtcgggctctccactcattacggtccacctcaagcacagattgacacgtaagcagcacagtgaggccaccaacagcagcaaccgagtcttcgtgtactgcgccttcctggacttcagctccggagaaggggtctggtcgaaccacggctgtgcgctcacgagaggaaacctcacctactccgtctgccgctgcactcacctcaccaactttgccatcctcatgcaggtggtcccgctggagcttgcacgcggacaccaggtggcgctgtcgtctatcagctatgtgggctgctccctctccgtgctctgcctggtggccacgctggtcaccttcgccgtgctgtcctccgtgagcaccatccggaaccagcgctaccacatccacgccaacctgtccttcgccgtgctggtggcccaggtcctgctgctcattagtttccgcctcgagccgggcacgaccccctgccaagtgatggccgtgctcctacactacttcttcctgagtgccttcgcatggatgctggtggaggggctgcacctctacagcatggtgatcaaggtctttgggtcggaggacagcaagcaccgttactactatgggatgggatggggttttcctcttctgatctgcatcatttcactgtcatttgccatggacagttacggaacaagcaacaattgctggctgtcgttggcgagtggcgccatctgggcctttgtagcccctgccctgtttgtcatcgtggtcaacattggcatcctcatcgctgtgaccagagtcatctcacagatcagcgccgacaactacaagatccatggagaccccagtgccttcaagttgacggccaaggcagtggccgtgctgctgcccatcctgggtacctcgtgggtctttggcgtgcttgctgtcaacggttgtgctgtggttttccagtacatgtttgccacgctcaactccctgcagggactgttcatattcctctttcattgtctcctgaattcagaggtgagagccgccttcaagcacaaaaccaaggtctggtcgctcacgagcagctccgcccgcacctccaacgcgaagcccttccactcggacctcatgaatgggacccggccaggcatggcctccaccaagctcagcccttgggacaagagcagccactctgcccaccgcgtcgacctgtcagccgtgtgagccgggaggctgccaaccaggccaggctgcgctcagaacacaccccccccaaacagaatgaaatgccccacctttgcccatggaccctctccttgctgctgtctggacatgggtgttgtggccccgagacagctgtcctcccctgtgactctggctgtcggagcacactgctcagcccagcagcctgatgcccaggccagcgtgggccctcctgccttgcatccacccgtgggctgagtgacttcctcgggggattcccaggacacagtggcctgactgtgatggtgcccttgagcctcccttcatcactcagcatcagacccagcgaggccaggacactcggggccggtcccgcagcaccaggaggggatgttcagcctctgtgccttggtggggcttggggactcagggccaaagaggtggttcaggtccccacgcaccctcagtcaggcgcaggcagctgggggtgtgtggggaagagcatgcggagtccccagtgtctgaatccactgagtggtgagttccccacagccggcgctagccgtggtgtgtgtctctgtaggtggtgccggcgtgggccaacctgtgctgtgtcatcagttgggggcccctgcccaagccgagctcgagccgtgggcgggagtcgttgactctccaggtgagggcgacccctctgccctgtccttgggggggtcccctctgctcacgtgaagagccgctctgggccttgaggctgcctgatggtgcctgtgcttgggggagcttctcggccatccgctgtgagttttgcctctttggaccccaattcggccttaagatgccctcctccctcgtgtgccagcctccttggttgttcttgggccacaggagctcgccgtgtccccgcagtgcctggtgtccaggtggaaagtggagggcattttccagggcactgctttccccagaggcttcctcatggctcacaggcactctacgaagtttctaatgggcagaccacgcggcaggtagcacagtgcgctccgtctggtcaccatgagaccgacctgcgctgagtccccactgacctggagagggagggctggtgacagccgtgtcttctgtgttgagggaaatttatggactcagactcagccccagaggagatgggataattgttatggacccatgtgtgggcatgatcctgtggaacacaggtttgggatcatagatgtgaattaagacaccaccgagatacgggctgtgaggttcataccgtgctgatagcactcgtggtgtctgtgaaatgtgggtaagacattcaaacctggttttgatactggaaactcttcctttaaaactgtgaccatgatttcattcagcccctccacacccctatgtctgccttgtttcagagtgagttttctatggagcctgtggcccttttgcagcccacctggtggcttcttaatgtaactcttcccctggtcgcctggagtggaccactcatctgcaggcctctcctgcatggggagggtaggcagggagcagcatgtctgcaggggtgaacctttgctcttctgtcaggcgaggcccaagctgcaccagccacctgccacatggtgacagtgccacgggccctgcgtatggcccctgcaaccgtgctctggcgggcacacctggctgctgcaggccaaggccgctgttcagtgaagagtcccatgtttagtatggactaaagtcccatgtttagccactgccccaggctcccgtgaccccagaaaccaggtcacatggaccacagtgccagatcctcatcacgccggtgagcacctagaagtgagaacactgtattcctacaatgtacacttggatatttctccttatttagtttctagtgaaacaaatcaagtaaggaactatctttagtttagatggaattatttgtttttaattgttgccgtattcatctatatagctaatatttcaagataagtaatgaacaaaacctgtctaaaccttttgtttccaatgaatgaaagtcatgcactttatttataggctctatgttttggcttctgcagtacttttattatctatacataatttggccaaaaataagaaattggaaagaatgaaatgtttagtttatagtagaagaaagatgatgacactaagttgtgaaaatatgttgtgatttttatgaaataaactcatgtcctg

SEQ ID NO: 6 - protein, 874 aa, Homo sapiens
MEKLLRLCCWYSWLLLFYYNFQVRGVYSRSQDHPGFQVLASASHYWPLENVDGIHELQDTTGDIVEGKVNKGIYLKEEKGVTLLYYGRYNSSCISKPEQCGPEGVTFSFFWKTQGEQSRPIPSAYGGQVISNGFKVCSSGGRGSVELYTRDNSMTWEASFSPPGPYWTHVLFTWKSKEGLKVYVNGTLSTSDPSGKVSRDYGESNVNLVIGSEQDQAKCYENGAFDEFIIWERALTPDEIAMYFTAAIGKHALLSSTLPSLFMTSTASPVMPTDAYHPIITNLTEERKTFQSPGVILSYLQNVSLSLPSKSLSEQTALNLTKTFLKAVGEILLLPGWIALSEDSAVVLSLIDTIDTVMGHVSSNLHGSTPQVTVEGSSAMAEFSVAKILPKTVNSSHYRFPAHGQSFIQIPHEAFHRHAWSTVVGLLYHSMHYYLNNIWPAHTKIAEAMHHQDCLLFATSHLISLEVSPPPTLSQNLSGSPLITVHLKHRLTRKQHSEATNSSNRVFVYCAFLDFSSGEGVWSNHGCALTRGNLTYSVCRCTHLTNFAILMQVVPLELARGHQVALSSISYVGCSLSVLCLVATLVTFAVLSSVSTIRNQRYHIHANLSFAVLVAQVLLLISFRLEPGTTPCQVMAVLLHYFFLSAFAWMLVEGLHLYSMVIKVFGSEDSKHRYYYGMGWGFPLLICIISLSFAMDSYGTSNNCWLSLASGAIWAFVAPALFVIVVNIGILIAVTRVISQISADNYKIHGDPSAFKLTAKAVAVLLPILGTSWVFGVLAVNGCAVVFQYMFATLNSLQGLFIFLFHCLLNSEVRAAFKHKTKVWSLTSSSARTSNAKPFHSDLMNGTRPGMASTKLSPWDKSSHSAHRVDLSAV